(19) United States
(12) Patent Application Publication
(10) Pub. No.: US 2007/0161697 A1
(43) Pub. Date: Jul. 12, 2007

(54) Hydrazide type compounds and the use thereof in pharmaceutical compositions for the treatment of cardiovascular diseases

(76) Inventors: Gerard Marguerie, Vitry-sur-Seine (FR); Eric Malaud, Nimes (FR)

Correspondence Address: YOUNG & THOMPSON, 745 SOUTH 23RD STREET, 2ND FLOOR, ARLINGTON, VA, 22202, US

(21) Appl. No.: 10/587,697

(22) PCT Filed: Jan. 31, 2005
(86) PCT No.: PCT/FR05/00199

§ 371 Date: Sep. 27, 2006

(30) Foreign Application Priority Data
Jan. 30, 2004 (FR) 0400913

(51) Int. Cl. A61K 31/405; A61K 31/381; A61K 31/34

(52) U.S. Cl. 514/419; 514/443; 514/614; 514/471; 548/492; 549/76; 549/57; 549/483

(57) ABSTRACT

The invention relates to compounds of general formula (I), where R1 and R2 are independently selected from a hydrogen atom, a branched or straight chain alkyl with 1 to 6 carbon atoms, a fluoroalkyl group with 1 to 6 carbon atoms and 3 to 7 fluorine atoms, A is an aromatic group with one or several rings, optionally comprising one or more heteroatoms and B is an optionally-substituted phenyl group, or an optionally-substituted pyridine.

The present invention concerns new hydrazide type compounds and their use as active agents in pharmaceutical compositions intended in particular for treatment or prevention of cardiovascular diseases.

In spite of highly active pharmacological research and major advances in the fields of surgery, cardiovascular diseases, coronary accidents and cerebral ischaemias remain the main cause of deaths and invalidities in the industrialised world. Type II diabetes mellitus and the metabolic syndrome associated with the latter, hypercholesterolaemia, obesity defined as an increase in fatty mass, hypertriglyceridaemia and atherogenic dyslipidaemia characterised by complex lipoprotein profiles constitute the recognised risk factors of these cardiovascular diseases.

These pathologies have in common a disorder of lipoprotein metabolism. The atherogenic dyslipidaemia of type II diabetes mellitus and the metabolic syndrome, for example, is characterised by a high level of triglycerides (greater than 150 mg/dl), a low high density lipoprotein cholesterol level (HDLc less than 40 mg/dl and a variable low density lipoprotein cholesterol (LDLc) level (less than or greater than 100 mg/dl). The hypertriglyceridaemia very often associated with obesity is characterised by a very high increase in the triglycerides (greater than 200 mg/dl) which enter into the structure of the lipoproteins.

The most serious complication of all these syndromes is atherothrombosis. Atherothrombosis is a complex disorder related to these metabolic disorders and the development of which is silent and gradual and may begin very early in life, involving several successive phases.

The formation of a lipid-rich arterial plaque is a slow process generally developing over several decades. It involves a gradual accumulation of lipoprotein, foamy macrophages and calcium on the arterial wall. The plaques affect the majority of individuals subject to the diet rich in animal fats of western industrialised countries, but a high degree of variability between individuals exists in the rate of evolution and extension of the plaques which is partially due to genetic characteristics.

The presence of numerous foamy macrophages in the plaque makes it vulnerable and causes episodes of rupture. Rupture of the atherosclerosis plaque and formation of a platelet thrombus are for their part acute processes responsible for the severe complications of the disease: coronary and cerebral infarction and sudden death. The severity of the disease therefore depends largely on the size of the plaque, its stability and the manner in which the thrombus is formed by rupture of this plaque. This phenomenon is rather poorly understood and often involves a chronic inflammatory state in addition to an immune response. To date, different therapeutic options are available for treatment of these diseases.

Hypolipaemic agents such as statins or ezetimibe possess recognised efficacy. Statins are inhibitors of 3-hydroxy-methylglutaryl coenzyme A reductase which is directly involved in cholesterol synthesis. Statins effectively reduce the cholesterol level and to a more limited degree, the triglyceride level. Ezetimibe inhibits intestinal absorption of cholesterol. These molecules are therefore recommended as primary and secondary prevention for the majority of patients with a high LDLc level. The clinical trials have shown however that the medical benefit of hypolipidaemic agents, with regard to the cardiovascular risk, is only 30 to 35%. Their use is sometimes accompanied by undesirable adverse events which require treatment withdrawal. In many cases, muscular involvement, hepatic toxicity and intolerance phenomena are observed.

Fibrates or fibric acid derivatives are also recommended for treatment of atherogenic dyslipidaemias. Dyslipidaemias affect different patients with complex lipid profiles: a low cholesterol level, high triglyceride levels and low HDLc levels. Use of fibrates reduces the risk of cardiovascular accidents by approximately 40%. Their use is unforturnately accompanied in many patients by undesirable effects due to intolerance, hepatic toxicity and muscle involvement.

The thrombotic accident resulting from rupture of an arterial plaque is generally treated with antithrombotic agents such as acetylsalicylic acid, thienopyridins or thyanopyridins.

New compounds are therefore needed which are capable of treating cardiovascular diseases and in particular in order to treat the growth and vulnerability of an arterial plaque.

The present invention precisely aims at new hydrazide type compounds used as an active agent in pharmaceutical compositions, especially for treatment or prevention of cardiovascular diseases.

The compounds of the invention correspond to the following general formula (I): 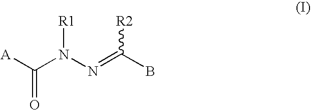
in which:
R1 and R2, identical or different, are chosen from among a hydrogen atom, a linear or branched lower alkyl radical of 1 to 6 carbon atoms, a fluoroalkyl radical of 1 to 9 carbon atoms and of 3 to 7 fluoride atoms,
A represents an aromatic group of one or several cycles possibly comprising one or several heteroatoms,
B represents a possibly substituted phenyl group or a possibly substituted pyridine group.

Owing to the hydrazide function in the double bond N═C and the meaning of the groups B and R2, the compounds of the invention of formula (I) may come in geometric forms known as (E) or (Z), existing either in equilibrium or preferentially in a single form (E):
form (E) in which the groups ACONR1 and B are on either side of the imine function N═C, known as the trans form, or
form (Z) in which the groups ACONR1 and B are on the same side of the imine function N═C, known as the cis form.

Preferred compounds of formula (I) are those in which B represents a group of the following formula (II) 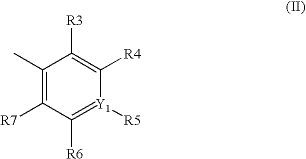
in which Y₁ is a carbon atom in order to form a phenyl nucleus or a nitrogen atom in order to form a pyridine nucleus and in which R3, R4, R5, R6 and R7, either identical or different, are chosen from among: an atom of hydrogen, an atom of halogen and more particularly of fluoride, chloride and bromide, a group of formula —OH, —OR8 or —OCOR9, in which R8 and R9 represent a linear or branched lower alkyl radical of 1 to 6 carbons, an amino group —NH₂ or —N(r, r′) in which r and r′, either identical or different, represent a linear or branched lower alky radical, an aryl radical, or a heterocycle in which r and r′, taken together, form a heterocycle of variable size, preferably in the para position.

Preferred compounds are those of formula (I) in which R3 is a group of formula —OR8 and at least two of the substituents R4, R5, R6 and R7 represent a hydrogen atom. Among the latter, one also especially prefers the compounds of formula (I) in which Y₁ is a carbon atom.

Other preferred compounds are those of formula (I) in which B is of formula (II) in which Y₁ is a carbon atom, corresponding to the following formula (I′):

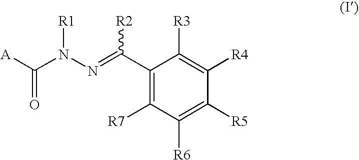

A first form of realisation of the invention relates to the compounds of formula (I) in which A represents a group of the following formula (III): 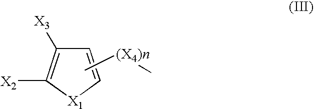
in which:
X₁ is chosen from among:
an oxygen atom and in this case the group of formula (III) is a 2-furanyl or 3-furanyl nucleus as a function of the position of the chain —(X₄)ₙ-acyl-hydrazide on the α or β carbons of this heterocycle,
a sulphur atom and in this case, the group of formula (III) is a 2-thiophene or 3-thiophen nucleus as a function of the position of the chain —(X₄)ₙ-acyl-hydrazide on the α or β carbons, this sulphur atom being capable of bearing an oxygen atom in order to form a sulphoxide or two oxygen atoms in order to form a sulphone.
a nitrogen atom and in this case, the group of formula (III) is a 2-pyrrol or 3-pyrrol nucleus as a function of the position of the acyl-hydrazide chain on the α or β carbons of this heterocycle, this nitrogen atom being capable of bearing a hydrogen atom, a lower alkyl radical of 1 to 6 carbon atoms, a fluoroalkyl radical with 1 to 6 carbon atoms and 3 to 7 fluoride atoms, an acyl radical —COR10 in which R10 represents a linear or branched alkyl chain of 1 to 6 carbons or an aryl or aralkyl radical,
an oxygen atom and in this case the group of formula (III) is an N-oxide.
X₂ and X₃, either identical or different, are chosen from among:
a hydrogen atom, a linear or branched lower alkyl chain of 1 to 6 carbon atoms, a fluoroalkyl radical with 1 to 6 carbon atoms and 3 to 7 fluoride atoms,
a halogen atom, preferentially a fluoride, chlorine or bromide atom,
a nitro —NO₂ group, an amino —NH₂ group or a —N (r, r′) group, in which r and r′, either identical or different represent a linear or branched lower alkyl radical, an aryl radical, or a heterocycle of variable size,
   or furthermore X₂ and X₃ are included in an aromatic benzenic or aza-benzenic type cycle if this cycle comprises a nitrogen atom, in order to form an aromatic benzofuran heterocycle when X₁ is an oxygen atom, a benzopyrrol nucleus when X₁ is a nitrogen atom either free or substituted as above, a benzothiophene nucleus when X₁ is a sulphur atom either free or substituted as above or furthermore a pyridino type nucleus if an intracyclic nitrogen atom is present,
n is 0 or 1,
X₄, if present, represents a —CH₂—, —OCH₂—, or —CH═CH— group.

Compounds of formula (I) in which B is a group of formula (II) and A is a group of formula (III) corresponding to the following formula (IV): 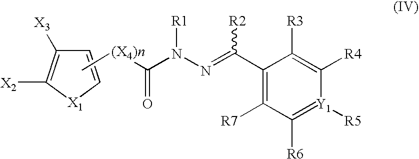
in which Y₁, X₂, X₃, R1 and R2 have the same meaning as above and R3 to R7, either identical or different, are chosen from among an atom of hydrogen, an atom of halogen and more particularly, of fluoride, chlorine and bromide, a group of formula —OH, —OR8 or —OCOR9, in which R8 and R9 represent a lIinear or branched lower alkyl radical of 1 to 6 carbons, an amino group —NH₂ or —N(r, r′) in which r and r′, either identical or different represent a linear or branched lower aLkyl radical, an aryl radical or a heterocycle in which r and r′, taken together, form a heterocycle of variable size, preferably in the para position.
n is 0 or 1,
X₄, if present, represents a —CH₂—, —OCH₂—, or —CH═CH— group.

Preferred compounds are those of formula (IV) in which R3 is a group of formula —OR8 and at least two of the substituents R4, R5, R6 and R7 represent a hydrogen atom. Among the later, one also especially prefers the compounds of formula (IV) in which Y₁ is a carbon atom.

Among the compouinds of formulas (IV), the invention refers more specifically to the following compounds:
N′-[(1E)-(2-hydroxy-4,6-dimethoxyphenyl)methylene]-1-benzothiophene-2-carbohydrazide (designated CGP02-01),
(2Z)-3-(2-furyl)-N′-[(1E)-(2-hydroxy-4,6-dimethoxyphenyl) methylene] acrylohydrazide (designated CGP02-02),
N′-[(1E)-(2-hydroxy-4,6-dimethoxyphenyl)methylene]-5-methylthiophene-2-carbohydrazide (designated CGP02-03),
2-furancarboxylic acid (2-hydroxy-4,6-dimethoxy-benzylidene)-hydrazide (designated CGP02-07),
(1H-indol-3-yl) acetic acid (2-hydroxy-4,6-dimethoxybenzylidene)-hydrazide (designated CGP02-08),
benzo[b]thiophene-2-carboxylic acid (3,5-dibromo-2-hydroxy-benzylidene)-hydrazide (designated CGP02-18).

Among these, one prefers in particular N′-[1E)-(2-hydroxy-4,6-dimethoxyphenyl)methylene]-1-benzothiophene-2-carbohydrazide (designated CGP02-01).

A second form of realisation of the invention concerns compounds of formula (I) in which A represents a group of the following formula (V): 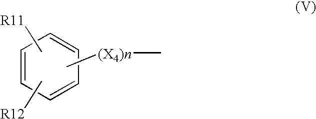
in which:
n is 0 or 1
X₄, if present, represents a —CH₂—, —OCH₂—, or —CH═CH— group.
R11 and R12, either identical or different, in the ortho, meta or para positions in relation to the bond witth —X₄— or in relation to the bond with —CO— when n is 0, are chosen from among: a linear or branched-chain lower alkyl or aralkyl group of 1 to 6 carbon atoms or a fluoroalkyl radical with 1 to 6 carbon atoms and 3 to 7 fluoride atoms, a —OH, —OR13 or R13 radical represents a linear or branched-chain lower alkyl group of 1 to 6 carbon atoms, a halogen and more particularly of fluoride and specifically in this case, when R11 and R12 are fluoride atoms, they are in ortho on either side of the bond with —X₄— or the remainder —CO—,
where R12 represents a hydrogen atom and R11 represents a type —SO₂NH₂ suphonamide group, in para in relation to the bond with —X₄— or the remainder —CO—,
or furthermore R11 represents a hydrogen atom and R12 represents a -Ophenyl group in ortho in relation to the bond with —X4— or the remainder —CO—,

Compounds of formula (I), in which B is a group of formula (fl) and A is a group of formula (V), correspond to the following formula (VI): 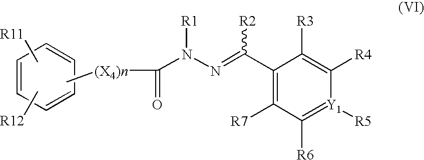
in which Y₁, X₄, R1, R2, R11 and R12 have the same meaning as above and R3, R4, R5, R6 and R7, either identical or different, are chosen from among a hydrogen atom, a halogen atom and an atom of hydrogen, an atom of halogen and more particularly of fluoride, chloride and bromide, a group of formula —OH, —OR8 or —OCOR9, in which R8 and R9 represent a linear or branched lower alkyl radical of 1 to 6 carbons, an amino group —NH₂ or —N(r, r′) in which r and r′, either identical or different, represent a linear or branched lower alky radical, an aryl radical, or a heterocycle in which r and r′, taken together, form a heterocycle of variable size, preferably in the para position.

Preferred compounds are those of formula (VI) in which R3 is a group of formula —OR8 and at least two of the substituents R4, R5, R6 and R7 represent a hydrogen atom. Among the latter, one also especially prefers the compounds of formula (VI) in which Y₁ is a carbon atom.

Among the compounds of formulas (VI), the invention refers more specifically to the following compounds:
(4-dimethylamino-N′-[(1E)-(2-hydroxy-4,6-dimethoxyphenyl)methylene]benzohydrazide (designated CGP02-04),
2-phenethylbenzoic acid (2-hydroxy-4,6-dimethoxy-benzylidene)-hydrazide (designated CGP02-05),
N-[3-2-hydroxy-4,6-dimenthoxy-benzylidene-hydrazinocarbonyl)-phenyl]-propionamide (designatled CGP02-06),
(3-Chloro-phenoxy)-acetic acid (2-hydroxy-4,6-dimethoxybenzylidene)-hydrazide (designated CGP02-09),
2-phenoxy-benzoic acid (2-hydroxy-4,6-dimethoxybenzylidene)-hydrazide (designated CGP02-11),
2,6-difluorobenzoic acid (2-hydroxy-4,6-dimethoxybenzylidene)-hydrazide (designated CGP02-13),
4-trifluoromethylbenzoic acid (2-hydroxy-4,6-dimethoxy-benzylidene)-hydrazide (designated CGP02-16).
3,4-dimethoxybenzoic acid (4-diethylamino-2-hydroxy-benzylidene)-hydrazide (designated CGP2-17)

A third form of realisation of the invention concerns compounds of formula (I) in which A represents a group with the following formula (VII): 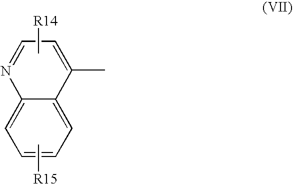
in which:
R15 is chosen from among an atom of hydrogen, an atom of halogen and more particularly of fluoride, chloride or bromide, a group of formula —OH, —OR16, in which R16 represents a linear or branched chain lower alkyl radical of 1 to 6 carbons or a fluoroalkyl radical with 1 to 6 carbon atoms and 3 to 7 fluoride atoms and more particularly a trifluoromethyl radical CF₃, R15 being positioned at one of the four remaining free sites of the 3-oxo-3,4-dihydro-benzothiazin-yl bicyclic aromatic part and
R14 represents a linear or branched alkyl radical of 1 to 6 carbons and more particularly a cyclopropy radical.

Compounds of formula (I) in which B is a group of formula (II) and A is a group of formula (VII) corresponding to the following formula (VIII): 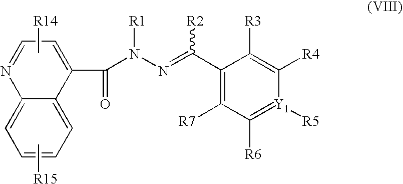
in which Y₁, R1, R2, R14 and R15 have the same meaning as above and R3, to R7, either identical or different, are chosen from among a hydrogen atom, a halogen atom and more particularly of fluoride, chloride and bromide, a group of formula —OH, —OR8 or —OCOR9, in which R8 and R9 represent a linear or branched lower alkyl radical of 1 to 6 carbons, an amino group —NH₂ or —N(r, r′) in which r and r′, either identical or different, represent a linear or branched lower alky radical, an aryl radical, or a heterocycle in which r and r′, taken together, form a heterocycle of variable size, preferably in the para position.

Preferred compounds are those of formula (VIII) in whicth R3 is a group of formula —OR8 and at least two of the substituents R4, R5, R6 and R7 represent a hydrogen atom. Among the latter, one also especially prefers the compounds of formula (VIII) in which Y₁ is a carbon atom.

Among the compounds of formula (VIII), the invention refers more specifically to the following compound:
2-cyclopropylquinoline-4-carboxylic acid (2-hydroxy-4,6-dimethoxy-benzylidene)-hydrazide (designated CGP02-14).

Preferred compounds are those of formula (I) in which R14 is in position 2 of the quinoline group and in which A represents a group of the following formula (VII′): 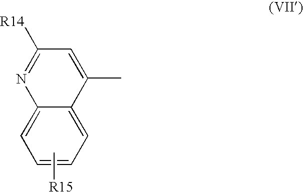
in which R14 and R15 have the same meaning as above.

Compounds of formula (I) in which B is a group of formula (II) and A is a group of (VII′) correspond to the foliowing formula (VIII′): 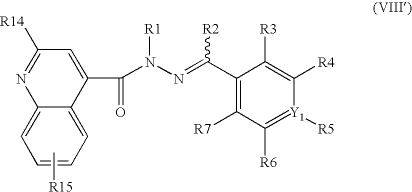
in which Y₁, R1, R2, R14 and R15 have the same meaning as above and R3, R4, R5, R6 and R7, either idenical or different, are chosen from among: a hydrogen atom, a halogen atom and more particularly of fluoride, chloride and bromide, a group of formula —OH, —OR8 or —OCOR9, in which R8 and R9 represent a linear or branched lower alkyl radical of 1 to 6 carbons, an amino group —NH₂ or —N(r, r′) in which r and r′, either identical or different, represent a linear or branched lower alky radical, an aryl radical, or a heterocycle in which r and r′, taken together, form a heterocycle of variable size, preferably in the para position.

Preferred compounds are those of formula (VIII′) in which R3 is a group of formula —OR8 and at least two of the substituents R4, R5, R6 and R7 represent a hydrogen atom.

Among the latter, one also especially prefers the compounds of formula (VIII′) in which Y₁ is a carbon atom.

A fourth form of realisation of the invention concerns compounds of formula (I) in which A represents a group of the following formula (IX): 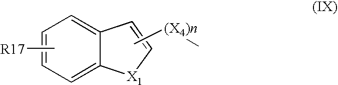
in which:
X₁ and X₄ have the same meaning as above,
n is 0 or 1,
R17 is chosen from among:
a hydrogen atom, a linear or branched lower alkyl radical of 1 to 6 carbon atoms, a fluoroalkyl radical of 1 to 6 carbon atoms and 3 to 7 fluoride atoms,
a halogen atom, preferentially an atom of fluoride, chlorine or bromide,
a group OR′ for which linear or branched lower R′ of 1 to 6 carbon atoms, a fluoroalkyl radical of 1 to 6 carbon atoms and 3 to 7 fluoride atoms.

Compounds of formula (I) in which B is a group of formula (II) and A is a group of formula (IX) correspond to the following formula (X): 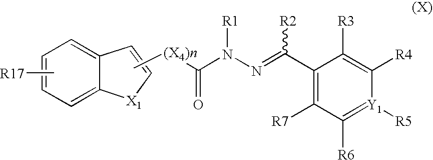
in which Y₁, R1, R2, R17, X₁, X₄ and n have the same meaning as above and R3, R4, R5, R6 and R7, either identical or different, are chosen from among a hydrogen atom, a halogen atom and more particularly of fluoride, chloride and bromide, a group of formula —OH, —OR8 or —OCOR9, in which R8 and R9 represent a linear or branched lower alkyl radical of 1 to 6 carbons, an amino group —NH₂ or —N(r, r′) in which r and r′, either identical or different, represent a linear or branched lower alky radical, an aryl radical, or a heterocycle in which r and r′, taken together, form a heterocycle of variable size, preferably in the para position.

Preferred compounds are those of formula (X) in which R3 is a group of formula —OR8 and at least two of the substituents R4, R5, R6 and R7 represent a hydrogen atom. Among the latter, one also especially prefers the compounds of formula (X) in which Y₁ is a carbon atom.

The invention also concerns, whenever possible, the salts of the above compounds with pharmaceutical type acids which are tolerated physiologically.

As an example of physiologically acceptable pharmaceutical salts, one may mention in a non-limitative manner, the salts of acetic, hydrochloric, cinnamic, citric, formic, hydrobromic, hydrolodic, hydrofluoric, malonic, methanesulphconic, oxalic, picric, maleic, lactic, nicotinic, phenylacetic, phosphoric, succinic and tartric acid, ammonium, diethylamine, piperazine, nicotinamide, urea, sodium, potassium, calcium, magnesium, zinc, lithium, methylamino, dimethlyamino, trimethylamino and tris(hydroxymethyl)aminomethane salts.

The invention concerns pharmaceutical compositions for humans or animals comprising as the active agent at least one of the compounds described above or their pharmaceutically acceptable salts.

Indeed, these compounds are useful for treatment of atherosclerosis and arterial restenosis. They possess the property of reducing weight gain due to accumulation of abdominal fat, of reducing the increase in the total cholesterol and free cholesterol level and deposit of triglycerides on the arterial wall and of reducing accumulation of macrophages in the atheromatous plaques. These compounds particularly possess the property of inhibiting formation of foamy macrophage cells by inhibiting accumulation of intracellular lipid vesicles. By extension, these molecules are therefore capable of treating obesity, type II diabetes mellitus, cerebral ischaemia and hepatic steatosis, by blocking accumulation of lipid vesicles in the cells such as hepatocytes, smooth muscle cells, adipocytes and endothelial cells.

These compounds are therefore useful as active agents in methods or pharmaceutical compositions for treatment and possibly prevention of all diseases associated with lipid metabolism. In this respect, one may mention, among others, hypercholesterolaemia, hypertriglyceridaemia, dyslipoproteinaemia, chylomicronaemia, lipodystrophy and hyperglycaemia, in addition to the disorders associated with these dysfunctions: atherosclerosis, obesity, type II diabetes mellitus or insulin resistance, heart failure and cerebral ischaemia (stroke).

Furthermore, since these compounds possess the properly of reducing stenosis of the arterial wall, they are useful as active agents in methods or pharmaceutical compositions for treatment and possibly prevention of restenosis.

The pharmaceutical compositions according to the invention comprise sufficient quantities of at least one compound described above.

Based on the results obtained in vivo and presented in the experimental section below, the compositions of the invention may be administered as part of treatment a several doses of 0.01 to 500 milligrams per day per kilogram of body weight of one or several compounds of the invention.

The formulation of the pharmaceutical compositions according to the invention is of the type generally used in the pharmaceutical field.

As an example, these may involve pharmaceutical vectors such as salts or electrolytes, salts of scorbic acid, water or buffered solutions, colloidal solutions, substances based on cellulose, polyethylene glycol, polyacrylates, waxes, proteins or any other substance capable of dissolving or rendering the active compound available for therapeutic action.

The compositions of the present invention may be administered in injectable form or via the oral or parenteral route, via the nasal route in spray form, via the rectal or vaginal route, by implantation of a reservoir or dispensers or in any other pharmaceutical form used in the pharmaceutical field.

The injectable forms of these compositions may be aqueous or oily suspensions. These suspensions may be formulated according to any process used in this field by using non-toxic solvents or diluents such as 1,3-butanediol for example. Among the acceptable solvents, it is possible to use water, buffered solutions, Ringer solutions, or isotonic salt solutions. Other acceptable diluents may be formed of synthetic mono or di-glycerides, long-chain alcohols, or dispersants such as carboxymethyl cellulose or any other diluent or emulsifier used in formation of pharmaceutical suspensions.

The pharmaceutical compositions of the present invention administered via the oral route may be in the form of capsules, tablets or aqueous suspensions or in the form of emulsions. These formulations may possibly contain chemical compounds intended to attenuate or improve the taste.

The pharmaceutical compositions of the present invention may be administered in suppository form by mixing the product with a non-irritant, non-allergic, excipient, solid at ambient temperature and liquid at rectal temperature in order to release the active compound. Such formulations may for example use beeswax, polyethylene glycols or cocoa butter.

These pharmaceutical compositions may also comprise a combination of one or several compounds of the invention with one or several other therapeutic molecules. These molecules may for example be hypolipaemic agents reducing cholesterol synthesis such as “statins”, angiotensin II converting enzyme inhibitors such as Losartan for example, anticalcium agents, antithrombotics , beta blockers inhibitors of the members of the class of peroxisome proliferator activated receptors (the PPAR class), inhibitors of triglyceride synthesis or metabolism such as the Fenofibrates, agents capable of increasing insulin resistance such as the Troglitazones or the Pioglitazones and generally speaking, any other molecule capable of improving the pharmacological performance of the compounds described in the present invention.

The invention also concerns use of a compound according to the invention for preparation of a pharmaceutical composition according to the invention.

The invention also concerns preparation of the compounds of formula (I) and pharmaceutical compositions containing at least one of the aforementioned compounds as the active ingredient.

The compounds of formula (I) may be prepared by the methods known to professionals in the field. The present invention describes in this respect a general route of synthesis illustrated by the scheme above and in the example of the following operating method in which the starting compounds are obtained commercially or may be synthesised according to the usual procedures known to professionals in the field and described in the conventional organic chemistry books (“Advanced Organic Chemistry” by M. B. Smith & J. March, Ed. John Wiley & Sons, “handbook of Heterocyclic Chemistry” by A. R. Katritzky, Ed. Pergamon and “Heterocyclic Chemistry” by J. A. Joule and K. Mills, Ed. Blackwell Science).

It is implied that the invention is not restricted to a specific route of synthesis and extends to other processes allowing production of the compounds of formula (I). As an example, the compounds of formula (I) may therefore be prepared either in a liquid phase or in a parallel phase on a solid medium. The methods given below are nonlimitative and any other procedures allowing creation of the double bonds of the substituted imine N═C type may be used in order to prepare the compounds of the invention.

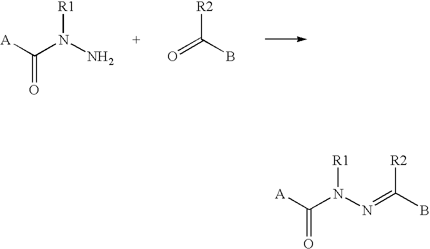

In the scheme above, R1, R2, A and B have the same meaning as above.

Accordcing to the scheme above, the compounds of the invention of formula (I) are directly prepared by a condensation reaction between on the one hand, the starting material designated carbo-hydrazide represented by the formula A—CO—NR1-NH2 and an aldehyde or a ketone represented by the formula R2-CO—B, for which the groups A and R1 on the one hand and B and R2 on the other hand have the meanings described for the formulas (II) to (VIII) respectively. These starting materials employed are commercial and may be obtained from chemistry companies working to order such as Maybridge (Great Britain) or Pfaltz-Bauer (USA), with this choice of companies not being exclusive.

This condensation reaction is preferentially conducted in an zinert atmosphere, between 0° C. and 50° C., preferably at ambient temperature in the presence of a tertiary amine organic base, preferably the base of Hüunig di-isopropylethylamine DIET, in an aprotic dipolar solvent, preferably anhydrous dimethylformamide DMF, or in ethanol under refoux for 6 to 8 hours. Monitoring of the progress of the reaction is performed by HPLC analysis, allowing control of the reaction time, preferably less than 24 hours.

Other advantages and characteristics of the invention will be apparent from the examples below and in which reference will be made to the drawings in the appendix in which:

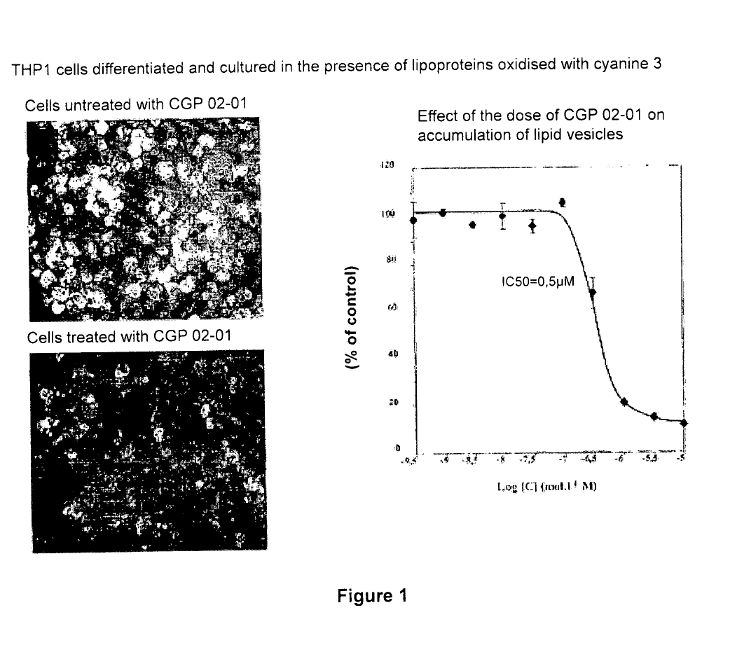

FIG. 1 represents the effects of increasing doses of the compound CGP02-0 on accumulation of lipid vesicles in a macrophage cell cultivated in the presence of lipoproteins marked using the fluorescent agent Cyanine 3. The dose response curve indicates a CI50 of 5×10⁻⁷ M.

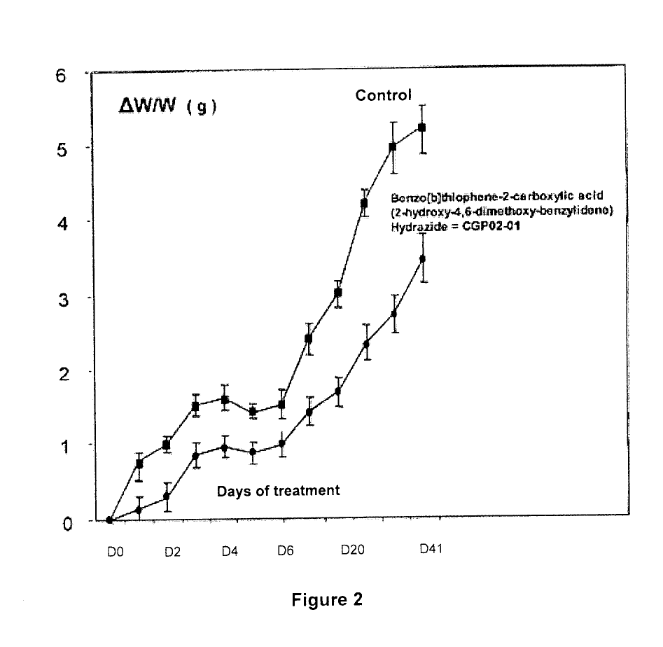

FIG. 2 shows the reduction in weight gain by reduction or abdominal fatty mass in ApoE negative mice following treatment with the compound CGP02-01. The mice were treated for 41 days at a dose of 20 μg of compound CGP02-01 per day. The control mice and the treated mice were fed with a normal dietary regimen without any cholesterol overload.

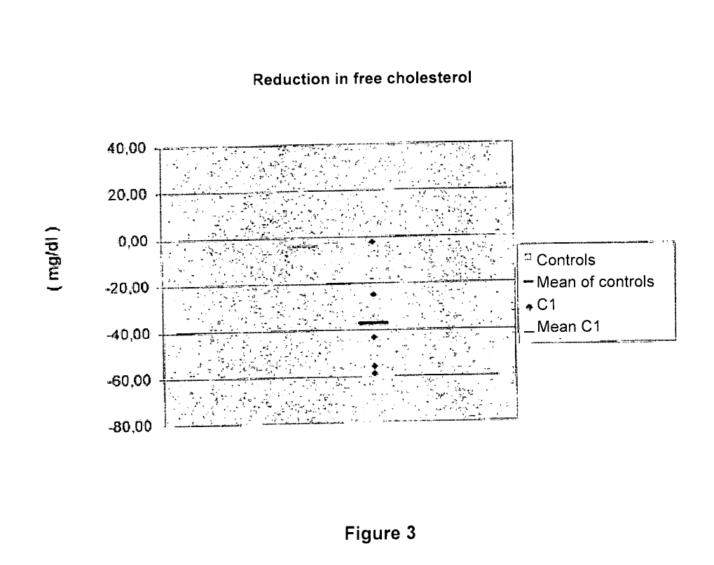

FIG. 3 represents the effect of the compound CGP02-01 on increase of the free cholesterol level in plasma in ApoE negative mice. The mice were treated in an identical manner to that described in FIG. 2.

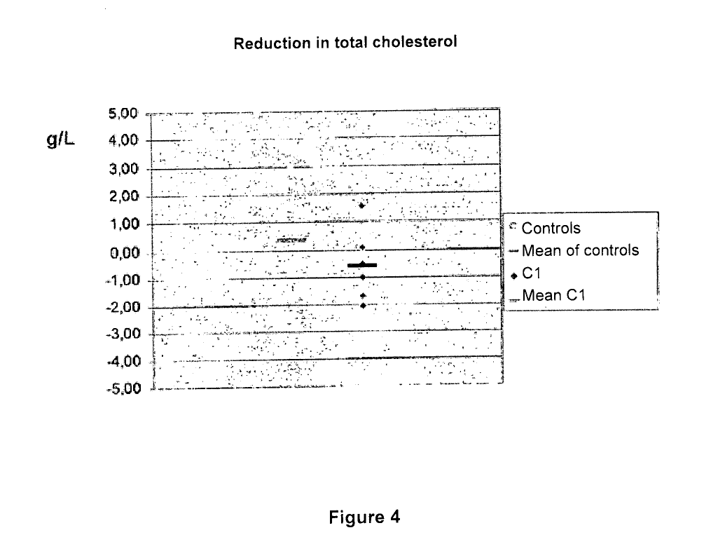

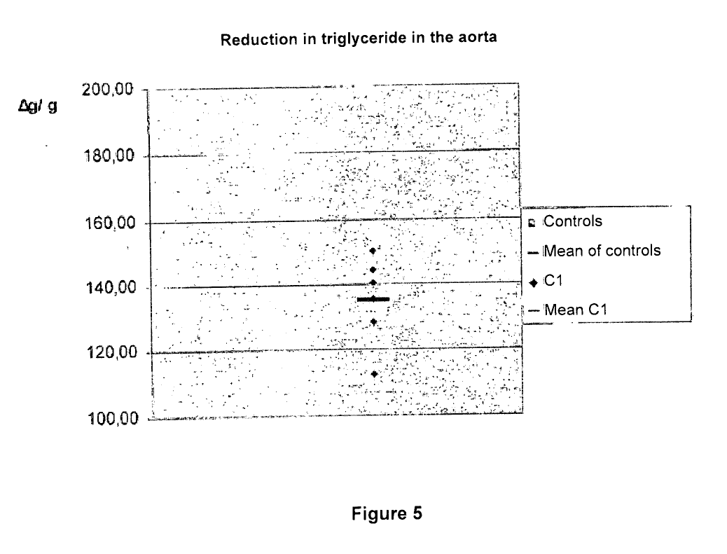

FIG. 4 shows the variation in the level of triglycerides present in the aorta of ApoE negative mice treated or untreated with the compound CGP02-01.

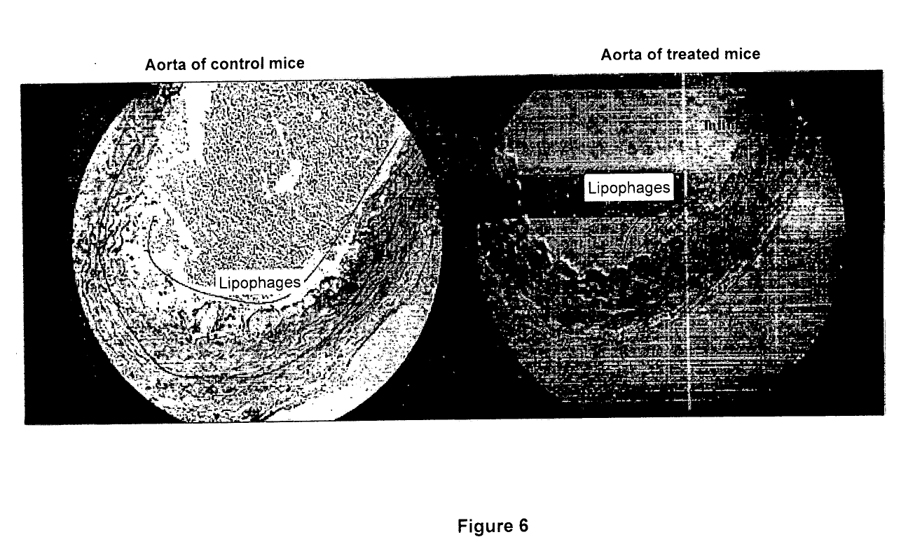

FIG. 6 shows the modification in the atheroma plaque in ApoE negative mice treated or untreated with the compound CGP02-01. The presence of an inflammatory situation is noted in addition to many foamy macrophages in the lesion of the untreated mice and a significant reduction in these macrophages in addition to an absence of an inflammatory reaction in the aorta of the treated mice.

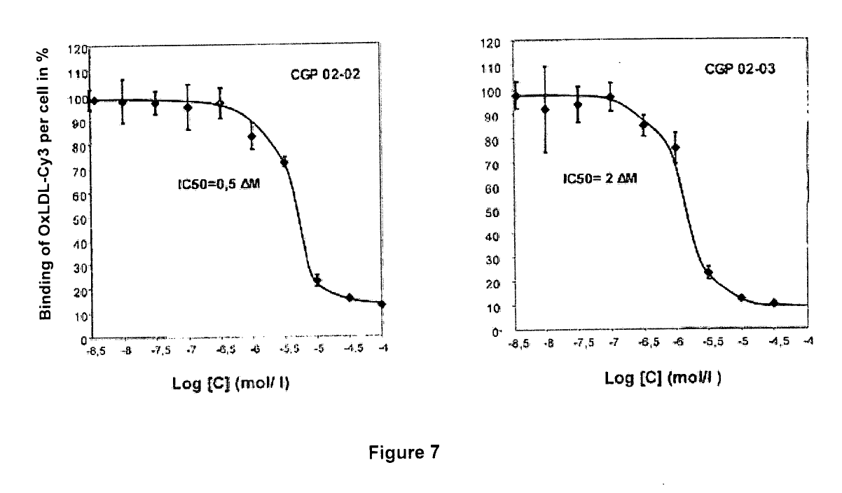

FIG. 7 shows inhibition of formation of foamy cells by the compounds CGP 02-02 and CGP 02-03. The differentiated THP1 cells are cultivated in the presence of oxidised lipoproteins (3 μg/ml oxLDL) marked with cyanine 3 for 25 hours at 37° C. The cells are treated with the compounds CGP 02-02 and CGP 02-03 at different concentrations. The conditions are those of FIG. 1.

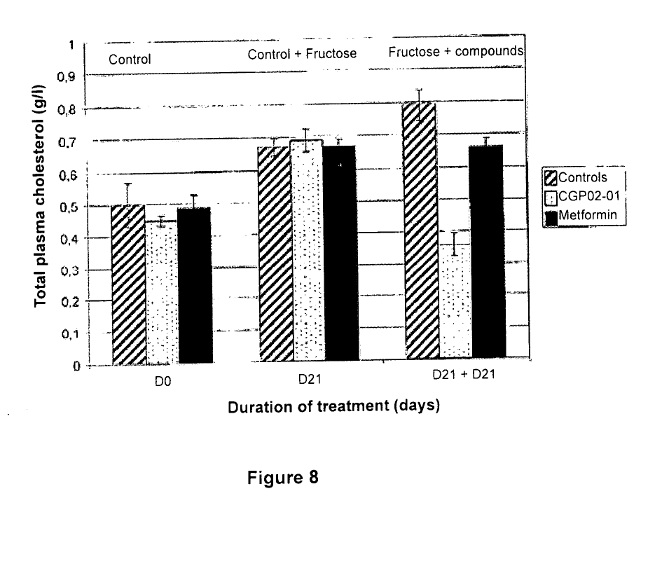

FIG. 8 represents the effect of the compound CGP 02-01 administered via the oral route (50 mg/kg) on the plasma concentration of total cholesterol (g/L) after 3 weeks of treatment of a rat model (n=12) subject to a fructose-rich dietary regimen (10%). Metformin was injected at the same dose to serve as a standard reference in this animal model.

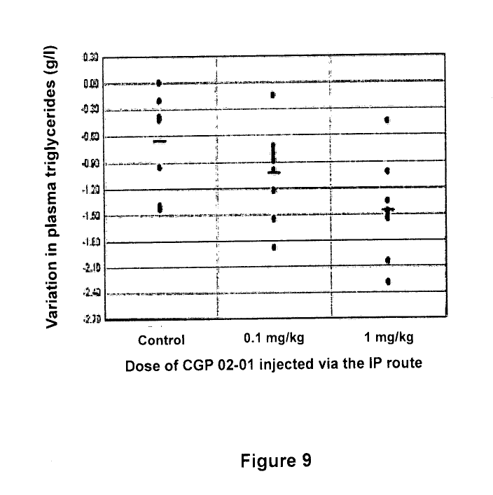

FIG. 9 illustrates the variation in the plasma triglyceride level (g/L) in an ApoE −/− mouse subjected to a dietary regime rich in cholesterol and treated for 3 months with the compound CGP 02-01.

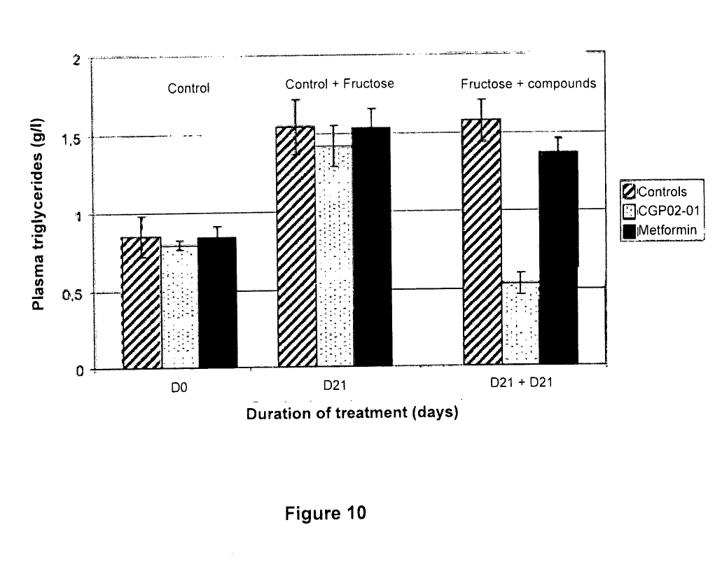

FIG. 10 shows the effect of the compound CGP 02-01 administered via the oral route (50 mg/kg) on the plasmatic level of triglycerides (g/L) after 3 weeks of treatment in a rat model (n=12), subjected to a dietary regimen rich in fructose. The regimen is subsequently maintained by adding the compound CGP 02-01 for 3 weeks. Each rat is analysed separately. Metformin was administered at the same dose in order to serve as a standard reference in this animal model.

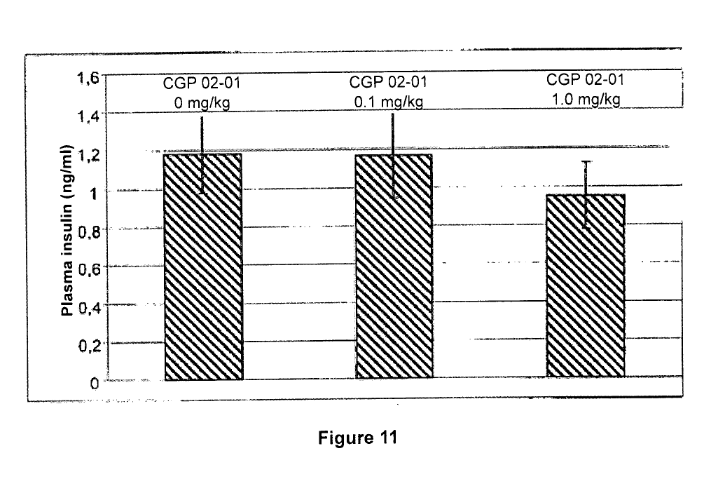

FIG. 11 represents the effect of the compound CGP 02-01 injected via the IP route on the plasmatic level of insulin (ng/mL) of ApoE −/− mice subjected to a dietary regimen rich in cholesterol. The mice are treated for 3 months.

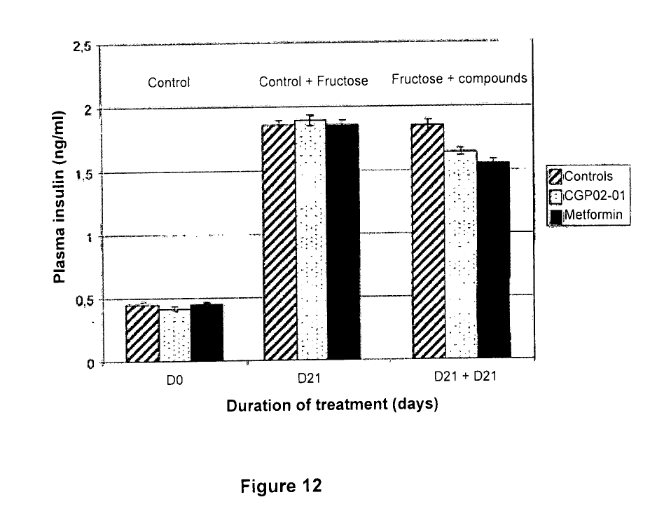

FIG. 12 shows the effect of the compound CGP 02-01 administered via the oral route (50 mg/kg) on the plasmatic level of insulin (ng/ml,) after 3 weeks of treatment in a rat model (n=12), subjected to a dietary regime rich in fructose (10%). The rats are fed daily for 3 weeks with a regimen containing 10% of fructose. The regimen is subsequently maintained by adding the compound CGP 02-01 for 3 weeks. Each rat is analysed separately. Metformin was administered at the same dose to serve as a standard reference in this animal model.

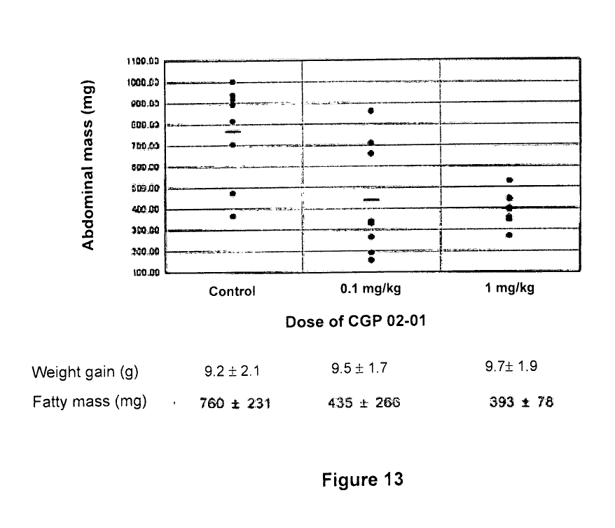

FIG. 13 shows the effect of the compound CCP 02-01. injected via the IP route at different doses on abdominal obesity of ApoE −/− mice subjected to a dietary regimen rich in cholesterol. The mice were treated for 3 months.

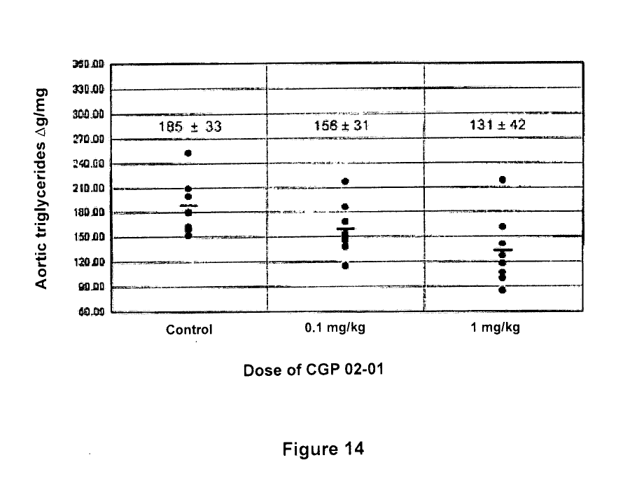

FIG. 14 shows the effect of the compound CGP 02-01 inject via the IP route at different doses on the deposit of triglycerides in the aortic wall of ApoE −/− mice subjected to a dietary regimen rich in cholesterol. The mice are treated for 3 months.

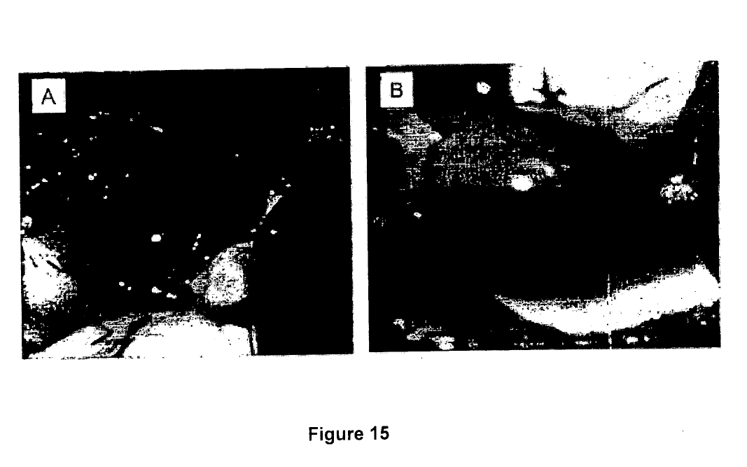

FIG. 15 shows that when ApoE −/− mice are subjected to a normal dietary regimen, or a dietary regimen rich in cholesterol, they develop coronary ischaemia illustrated by the presence of micro-embolisation of the coronary microvesssels (FIG. A). When the compound CGP 02-01 is injected in these mice, these cardiac lesions are considerably reduced (FIG. B).

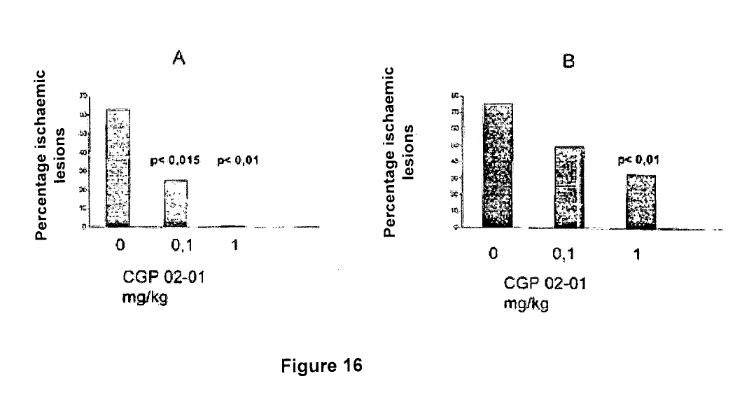

FIG. 16 illustrated the dose effect of the compound CGP 02-01 on the coronary lesions of ApoE −/− mice subjected to a normal dietary regimen (FIG. A) and a regimen rich in cholesterol (FIG. B).

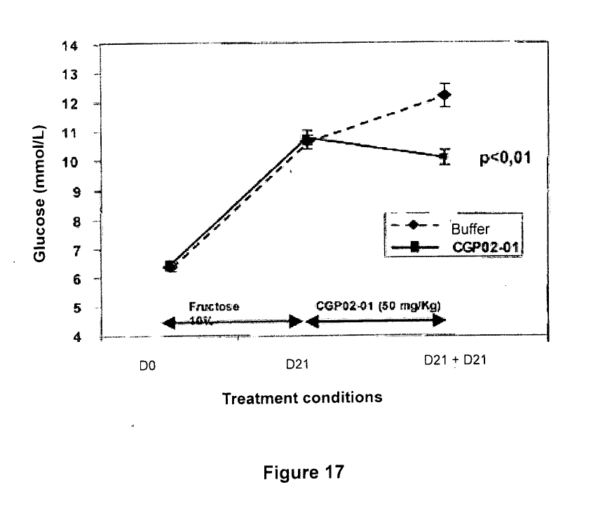

FIG. 17 illustrates the effect of the compound CGP 02-01 on the increase in glycaemia in rats subjected to a dietary regimen rich in fructose (10%). The regimen is maintained for 21 days. The compound is administered by the oral route following the period of the fructose diet. The figure illustrates the stabilising effect of the compound.

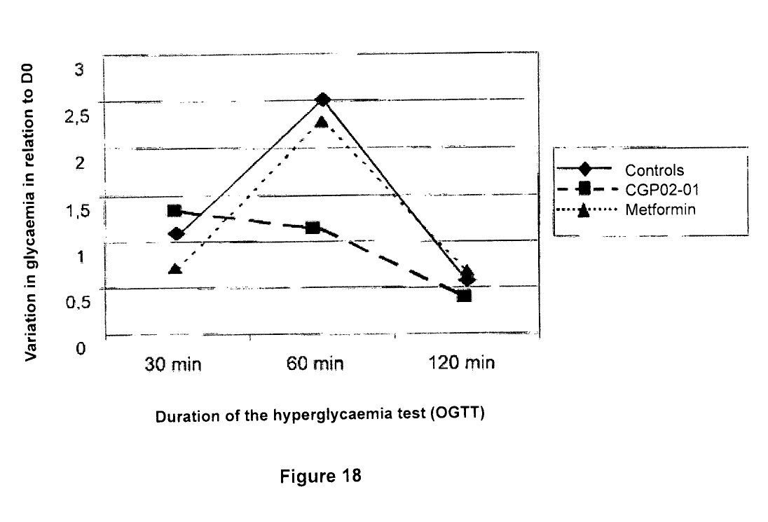

FIG. 18 illustrates the beneficial effect of the compound CGP 02-01 on glucose tolerance in rats subjected to a dietary regimen rich in fructose (10%). The compound is administered via the oral route and after the 14^{th} day, a single additional dose of 2 g/kg of glucose is administered. Glycaemia is measured at 30, 60 and 120 minutes after this glycaemic shock.

### EXAMPLE 1

Synthesis of CGP02-01.

Into a dry tricol flask equipped with magnetic stirring, 510.08 mg of commercial acid [benzo(b)thiophene]-2-carboxylic hydrazide dissolved in 28 ml of anhydrous DMF is introduced. After addition of 256 μl of DIEA (diethylisopropylamine), the solution is stirred for 5 minutes at ambient temperature. To this slightly yellow coloured solution, 538.27 mg of 4,6-dimethosysalicyl-aldehyde is added and the medium is stirred at ambient temperature for 24 hours. The progress of the reaction is monitored by HPLC analysis until complete consumption of the starting material. After evaporation of the solvent, the solid residue obtained is recrystallised in CH₃CN and subsequently washed with ethylic ether. The purified product obtained N′-[(1E)-(2-hydroxy-4,6-dimethoxyphenyl)methylene]-1-benzothiophene-2-carbohydrazide is a yellow solid (743.6 mg, yield=71%).

Physical and Chemical Characteristics:

Molecular mass: 356.40 g/mol

Melting point: 205.4° C.

LC-MS purity: 100% (M+1=357.33)

HPLC purity: 95.8% (retention time: 20 min, UV detection: 200-900 nm)

RMN 1H (DMSO-d6; 400 MHz): δ (ppm) 3.799 (s, 3H, OCH3), 3.862 (s, 3H, OCH3), 6.16 (s, 1H, Ar), 6.17 (s, 1H, Ar) 7.495 (m, 2H, Ar), 8.02 (dd, 1H, J=7.2 Hz and 1.3 Hz, 8.07 (dd, 1H, Ar, J=7.2 Hz and 1.4 Hz), 8.231 (s, 1H, CH═C), 8.861 (s, 1H, CH═N), 12.26 (s, 1H, CH═N), 12.26 (s, 1H, OH), 12. 348 (s, 1H, N—NH—CO).

NMR C13 (DMSO-d6, 400 MHz): δ (ppm) 55.438 (OCH3), 55.948 (OCH3), 90.524 (CH, Ar), 93.843 (CH, Ar), 122.846 (CH, Ar), 125.097 (CH, Ar), 124.439 (2CH, Ar), 125.684 (CH—C) 1.26.577 (CH═N), 145.980 (CO—NH═N).

IR-FT (KBr 0.05%) 3445.66 (Ar—OH) 1630.21 (—CO—NH═N), 1600.27 (—NH—N═C—) cm⁻¹.

Elemental analysis: C₁₈H₁₆N₂O₄S+0.5 H₂O

| | % C | % H | % N | % S |
|---|---|---|---|---|
| Theoretical | 59.17 | 4.69 | 7.67 | 8.77 |
| Found | 59.40 | 4.66 | 7.83 | 8.79 |

### EXAMPLE 2

Cell Cultures

Several lines of permanent cells may be used in order to demonstrate the effect of the molecules of the cass to which the molecule CGP02-01 belongs on the binding and accumulation of lipids in intracellular vesicles. These cells may incorporate lipoproteins, modifed lipoproteins, oxidised or acetylated for example, triglycerides or chylomicrons. These cells are able to transform themselves into foamy cells and may therefore present an atherogenic phenotype. It is posssible to use, as an example, THP1, U937, KG1 cells or any other cell capable of being activated and differentiated as macrophage, endothelial cell, smooth muscle cell hepatocyte or adipocyte and subsequently cultured in the presence of a medium containing lipoproteins.

Other types of cells having been genetically modified in order to express specific membrane receptors of binding of lipoproteins or fatty acids may also be used. These membrane receptors may form part of the family of the scavenger molecules containing proteins such as SRAI, SRAII, SRBI, CD36 or members of the family of the fatty acid receptors (FABP).

As an example, one may more specifically mention cells of the THP1 cell type differentiated under the action of phorbol 12-myristate-13-acetate (PMA) at a concentration of 10⁻⁷ M, which were used in order to measure the formation and accumulation of lipid vesicles observed during the formation of foamy macrophages in presence or absence of the compound CGP02-01.

The cells are cultivated in 96-well plates, at a density of 1, 2 or 5×10⁵ cells per ml in RPMI-1640 medium or in MEM medium containing 1%, 2%, 5% or 10% of foetal calf serum (FCS), 100 unit/ml of penicillin, 100 μg/ml of streptomycin, 200 mM of L-Glutamine at 37° C. in a CO2 incubator. The culture medium may be replaced every two days.

In the present example, the accumulation of lipid vesicles within the cell was measured using THP1 cells following fixation with paraformaldehyde in PBS medium using a solution containing a fluorescent marker of the Oil Red O type in order to visualise the vesicles. The image of the cells rich in vesicles was analysed using a microscope ecqluipped with a CCD camera and software necessary for the analysis.

The THP1 cells (5.10⁵ cells/ml) (ECACC) were maintained and cultivated in RPMI-1640 medium containing 10% of foetal calf serum (FCS), 200 mM of L-Glutamine, 100 units/ml of penicillin and 100 μg/ml of Streptomycin (Invitrogen-Life Technologies) at 37° C., in an incubator with 5% CO2. The medium was replaced every 2-3 days.

In order to induce differentiation of the THP-1, 1.25 10⁵ cells/well were deposited in the wells of a 96-well culture plate, in their culture medium containing 10⁻⁷ of phorbol 12-myristate-13-acetate (Sigma), for 24 hours at 37° C., 5% CO2. The differentiated THP-1 were subsequently incubated with LDLox coupled with cyanine-3 (1.5 μg/ml) in the presence or absence of the molecule CGP02-01 (concentrations of between 10⁻⁵M and 3.1.6 10⁻¹⁰M) for 24 hours at 37° C., 5% CO2. After fixation of the cells with 4% paraformaldehyde, the nuclei were marked with Hoechst 33342 (10 μg/ml) for 20 minutes at ambient temperature. After two washings, 16 images/well of the signal related to cyanine-3 and Hoechst 33342 were taken using a fluorescence microscope coupled with a CCD camera. Each image was analysed and quantified with the MetaMorph software (Universal Imaging).

Table 1 below reports the percentage observed for inhibition of the binding and accumulation in the form of lipid vesicles of lipoproteins marked with cyanine 3, by cells expressing the scavenger CD36. The cells were incubated in the presence of each of the molecules constituting this class and represented by the molecule CGP02-01 at a final and identical concentration for each molecule of 2.5 μM.

TABLE 1

| | Inhibition of accumulation of |
|---|---|
| Molecules | LDLox in (%) |
| CGP02-01 | 76 |
| CGP02-02 | 64 |
| CGP02-03 | 82 |
| CGP02-04 | 61 |
| CGP02-05 | 77 |
| CGP02-06 | 71 |
| CGP02-07 | 63 |
| CGP02-08 | 58 |
| CGP02-09 | 53 |
| CGP02-10 | 52 |
| CGP02-11 | 44 |
| CGP02-12 | 43 |
| CGP02-13 | 68 |
| CGP02-14 | 81 |
| CGP02-15 | 40 |
| CGP02-16 | 77 |
| CGP02-17 | 88 |
| CGP02-18 | 78 |

The table in appendix 1 gives the structures of the compounds of the invention in addition to their code in relation to table 1 above and likewise their percentage inhibition at a concentration of 25 microM on the THP1 cells for 24 hours.

One of the preferred compounds of this class is the compound CGP 02-01. When the differentiated type THP1 macrophage cells are cultivated in the presence of this compound at a concentration of 1 μM, one observes major inhibition of the accumulation o fipid vesicles (FIG. 1). This inhibiting effect depends on the concentration of the product CGP02-01 (FIG. 1).

Although the compound CGP 02-01 has been selected as a reference product, other compounds of the same class show the same activity and are capable of blocking accumulaLion of intracellular lipids. FIG. 7 illustrates the inhibiting activity of the compounds CGP 02-02 and CGI 02-03 forming part of the same class of molecules.

### EXAMPLE 3

Treatment of Atheromatous Mice.

Different types of animals may be used in order to study modifications in lipid metabolism, formation of arterial lesions and progression of an atheromatous plaque. These animals are commercially available. It is possible to use mice, rats, hyperlipaemic rabbits (HWWL) or larger animals such as pigs or monkeys. Genetically modified animals may also be used, such as ApoE −/−, LDL-R −/− and ApoAI −/− mice for example.

Two types of animal model have been used.

Firstly, mice devoid of the coding gene for Apo lipoprotein E (ApoE −/−) have been used. These mice represent a model of choice of studying early atherosclerosis and development of a plaque rich in foamy macrophages. Male C57BL/6J mice homozygote for deletion of the ApoE gene were subjected to a normal dietary regimen until the age of eight weeks. These mice (n=8 per group) subsequently received ad libitum and for 12 weeks either a dietary regimen not enriched with cholesterol or fat, or an enriched regimen, containing 1.5 g/kg of cholesterol and 200 g/kg of fat of milk origin. The untreated mice underwent daily intraperitoneal injections of a solution containing DMSO at 10%. The treated mice received by intraperitoneal injection the same solution containing 2 or 20 μg (i.e. 0.1 mg/kg/day or 1 mg/kg/day) of the compound CGP 02-01. Following the blood sample for the biochemical analyses, the mice were killed.

Secondly, the activity of the compound CGP 02-01 was studied using a reference rat-fructose model. Groups of Wistar rats (n=12) were subjected for 3 weeks to a dietary regimen containing 1.0% of fructose. Under these conditions, the rats develop a metabolic syndrome comprising hyperglycaemia, hyperinsulinaemia, hypercholesterolaemia and hypertriglyceridaemia. Groups of rats subsequently received by forced feeding 50 mg/kg of the compound CGP 02-01 dissolved in a 2% tween solution prepared in methylcellulose. The blood samples for the biochemical analyses were performed after 1, 2 and 3 weeks. Independent groups of rats wee treated under the same conditions with metformin hydrochloride at a dose of 50 mg/kg. The metformin served as a reference in this metabolic syndrome model.

### EXAMPLE 4

Measurement of Free Cholesterol in Plasma.

Measurement of free cholesterol may be performed by an enzymatic method. The free cholesterol is oxidised by cholesterol oxidase into Delta 4 cholestenon and simultaneously produces hydrogen peroxide. The hydrogen peroxide subsequently allows oxidative condensation of the DHESA and the aminoantipyrinre, producing a blue colour. The quantity of free cholesterol is subsequently measured by the absorbance of the blue colour. The samples may be recovered on citrate buffer containing EDTA and heparin. This test may be obtained commercially in kit form. When ApoE −/− mice are subjected to a normal non-enriched dietary regimen and are treated with the compound CGP 02-01 (1 mg/kg), the plasma level of non-esterified cholesterol significantly decreases. The variation observed in the untreated animals over a period of 3 months if 136±19 g/L on average. The variation observed for the animals treated for the same period is 105±6 g/L, i.e. an effect of 22.7% (p<0.05).

### EXAMPLE 5

Measurement of Total Cholesterol in Plasma

The circulating total cholesterol may be measured by enzymatic assay using the commercially available kit. This assay may for example use an enzymatic sequence of the cholesterol-esterase/cholesteroloxydase/chromogenic peroxidase type. In summary, the total esterified cholesterol is transformed into free cholesterol and fatty acid by the action of cholesterol esterase. The non-esterified cholesterol is subsequently measured by the formation of quinoeimine in the presence of cholesterol oxidase and peroxidise. The intensity of the quinoneimine coloration is proportional to the quantity of cholesterol present in the sample.

Table 2 below shows the variations in the plasmatic levels of HDL, and total cholesterol in an ApoE −/− mouse subjected to a dietary regimen rich in cholesterol and treated with the compound CGP 02-01. When ApoE −/− mice are treated with CGP 02-01 (1 mg/kg) for 3 months, the plasmatic level of total cholesterol decreases more significantly than in untreated animals. When mice are subjected to a dietary regimen rich in cholesterol, the total cholesterol level decreases from a value of 7.27±0.55 g/L to a value of 6.86±0.65 g/L, i.e. a variation of 5.6%. This effect observed is dependent on the dose of CGP 02-01.

TABLE 2

| | Plasma | Plasma total |
|---|---|---|
| | HDL level | cholesterol level |
| | (g/L) | (g/L) |
| Untreated | 0.11 ± 0.05 | 7.27 ± 0.55 |
| 0.4 mg/kg | 0.15 ± 0.06 | 8.08 ± 0.62 |
| 1 mg/kg | 0.15 ± 0.05 | 6.86 ± 0.65 |

In the model of rats subjected to a dietary regimen rich in fructose, treated via the oral route (50 mg/kg), the compound CGP 02-01 produces a highly significant reduction (p<0.01) in the total cholesterol level which decreases from a value of 0.79±0.05 g/L to a value of 0.36±0.03 g/L, i.e. a reduction of 54.5% (p<0.01) after 3 weeks of treatment. Metformin administered via the oral route under the same conditions (50 mg/kg) results in a reduction in total cholesterol of 16% with a mean value of 0.66±0.02 g/L (FIG. 12).

### EXAMPLE 6

Measurement of Circulating Triglycerides.

Assay of serum triglycerides may be performed enzymatically using the commercially available kit. One may use kits from Biomérieux for example (ref. 61.238). In summary, the triglycerides are treated with a lipase in order to generate fatty acids. In the presence of ATP, the glycerol is transformed by glycerokinase into glycerol 3 phosphate. The glycerol 3 phosphate is subsequently transformed into dihydroxyacetone in generating oxygenated water (H202) which may be detected by formation of quinoneimine in the presence of parachlorophenol, amino-4-antipyrin and peroxidase. The intensity of the quinoneimine coloration is subsequently measured at 505 nm. This coloration is proportional to the quantity of triglycerides present in the sample.

When ApoE −/− mice are subjected to a dietary regimen rich in cholesterol and in fat and are treated for 3 months with the compotund CGP 02-01 (1 mg/kg), their plasma triglyceride level varies and this variation is twice as great for the treated mice in relation to the untreated mice. This variation decreases from −0.67±0.54 g/L for the untreated mice to −49±0.57 g/L (p<0.01) for the treated mice (FIG. 9).

When the compound CGP 02-01 is administered via the oral route to rats under a dietary regimen rich in fructose, their plasma triglyceride level decreases from 1.39±0.13 g/L to 0.47±0.07 g/L, i.e. a variation of 66.2% (p<0.01). Under the same conditions, metformin does not have any effect, with a mean value of 1.21±0.08 g/L (FIG. 10)

### EXAMPLE 7

Measurement of Insulin in Plasma

Assay of insulin in plasma may be performed by radioimmunological assay using commercially available kits comprising specific anti-insulin antibodies of mice or rats. One may for example use the rat/mouse ELISA kits, Linco research (ref. EZRMI-13K).

When ApoE −/− mice subjected to a dietary regimen rich in cholesterol are treated with the compound CGP 02-01 at doses of 0.1 or 1 mg/kg, a significant reduction (p<0.01) in the plasma insulin level is observed, which decreases from a value of 1.17±0.2 ng/ml to a value of 0.95±0.16, i.e. a variation of 18.8% (FIG. 11).

In the same manner, the compound CGP 02-01 causes the insulin level of rats subjected to a dietary regimen rich in fructose to decrease from the value 1.85±0.04 to a value of 1.64±0.03, i.e. a variation of 11.3%. Under the same conditions, metformin results in a 16.2% reduction in the insulin level (FIG. 12).

### EXAMPLE 8

Measurement of the HDL Level in Plasma

The plasma HDL level is measured by tried and trusted commercial methods which use high density lipoprotein separation reagents and by measurement of the cholesterol level associated with these high molecular weight lipoproteins (Biomérieux kit ref. 61533 for example).

When mice subjected to a dietary regimen rich in cholesterol and fatty acid are treated with the compound CGP 02-01, their plasma HDL level increases from 0.11±0.005 g/L to 0.15±0.005 g/L, i.e. a variation of 38.2%.

### EXAMPLE 9

Measurement of Abdominal Fatty Mass

ApoE mice subjected to a dietary regimen rich in cholesterol and fat were killed after 3 months of treatment with the compound CGP 02-01 at doses of 0.1 mg/kg and 1 mg/kg. The abdominal fatty mass was recovered by dissection, dried and expressed as dry weight.

The compound CGP 02-01 results in a significant reduction (p<0.01) in abdominal fatty mass for a constant weight gain This abdominal mass decreases from a value of 760±231 mg to a value of 393±78 mg when the mice are treated with 1 mg/kg, i.e. a 48.3% reduction (FIG. 13). This effect depends on the dose of CGP 02-01.

### EXAMPLE 10

Triglyceride Deposit in the Aortas

The triglycerides which accumulate in the aortic wall were measured in the following manner: the aortas of the animals are rinsed with physiological saline after dissection. The lipid mass of the adventitia is eliminated by dissection and the intima media is dehydrated. The triglyceride level is measured and expressed as the weight of triglycerides per dry weight of tissue.

When ApoE −/− mice are treated with the compound CGP 02-01 for three months at a dose of 1 mg/kg, the triglyceride deposit on the aortic wall decreases from a mean value (n=8) of 185±33 μg/mg (dry weight) to a value of 131±42 μg/mg (dry weight). This effect depends on the dose injected. An injection of 0.1 mg/kg results in an intermediate variation of 156±31 μg/mg. FIG. 14 illustrates this result.

### EXAMPLE 11

Analysis of the Aortic Lesions

The aortas of the mice are fixed with paraformaldehyde and are dissected into 10 μm sections for histological analysis of the lesions (FIG. 6).

### EXAMPLE 12

Analysis of Coronary Ischaemia

After killing the ApoE −/− mice subjected to a normal dietary regimen or a dietary regimen rich in cholesterol, the hearts of these animals are observed macroscopically. The rate of ischaemic lesions is observed (FIG. 15) and quantified by the presence or absence of lesions (FIG. 16).

### EXAMPLE 13

Measurement of the Plasma Glucose Level in a Diabetic Rat Model

Glycaemia is measured by the hexokinase method using commercially available kits. The Biomérieux kit (ref.: 61 269/61 270 may be used for example.

When rats are subjected to a dietary regimen rich in fructose, their glycaemia increases as a function of time and increases from a mean value (n=12) of 6.4±0.15 mmole/L to a mean value of 10.65±0.24 mmole/L (FIG. 14). The compound CGP 02-01 stabilises this increase in glycaemia after 21 days of treatment via the oral route (FIG. 17)

### EXAMPLE 14

Measurement of the Protective Effect during a Hyperglycaemia Test

Rats (n=12) were subjected to a dietary regimen rich in fructose (10%) for 21 days. After the 14^{th} day, hyperglycaemic shock is induced by administration of 2 g/kg of glucose in the presence or absence of the compound CGP 02-01 administered via the oral route. The plasma glucose level is subsequently measured. FIG. 18 illustrates the protective effect of the product against the induced hyperglycaemia.

| | | Inhibition at |
|---|---|---|
| | | 2.5 μM on THP-1 |
| Structure | Compounds | cells (24 hours) |
| 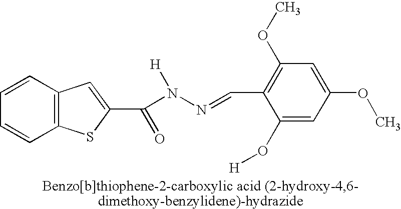 | CGP02-01 | 76% |
| 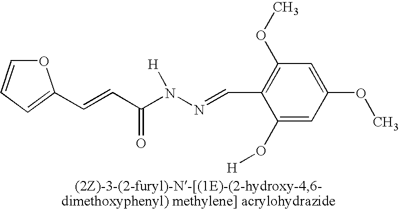 | CGP02-02 | 64% |
| 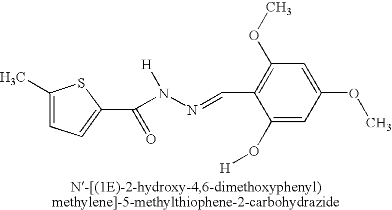 | CGP02-03 | 82% |
| 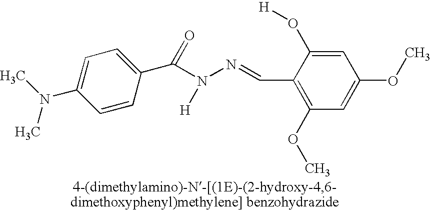 | CGP02-04 | 61% |
| 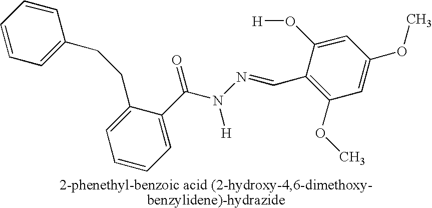 | CGP02-05 | 77% |
| 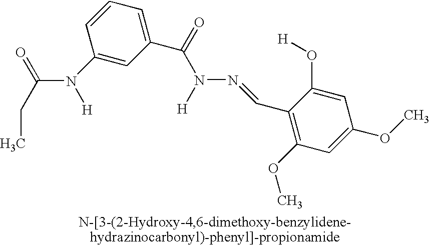 | CGP02-06 | 71% |
| 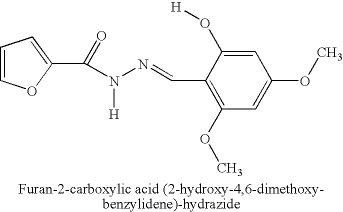 | CGP02-07 | 63% |
| 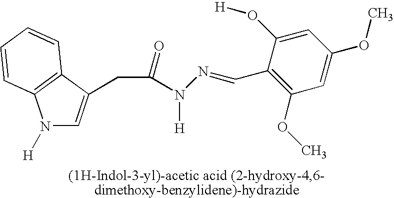 | CGP02-08 | 58% |
| 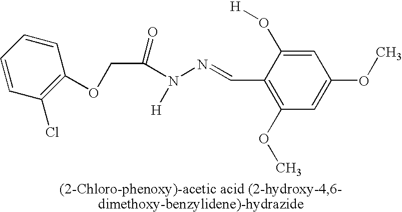 | CGP02-09 | 53% |
| 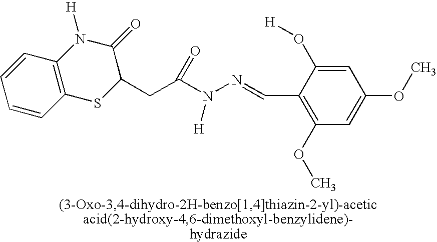 | CGP02-10 | 52% |
| 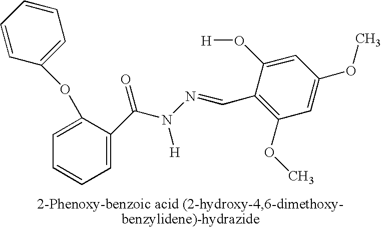 | CGP02-11 | 44% |
| 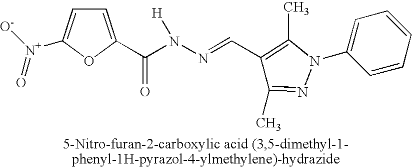 | CGP02-12 | 43% |
| 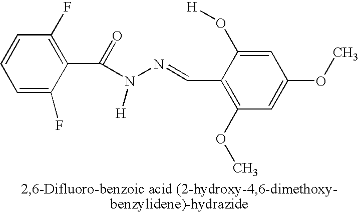 | CGP02-13 | 68% |
| 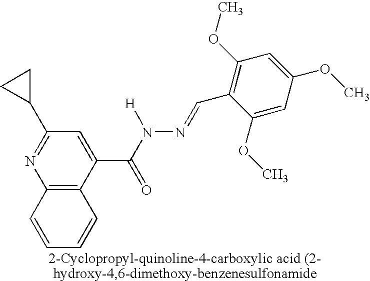 | CGP02-14 | 81% |
| 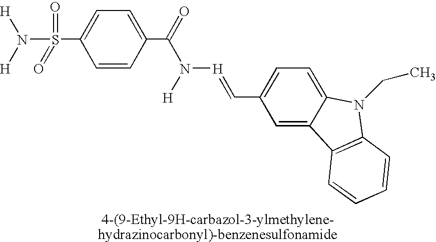 | CGP02-15 | 40% |
| 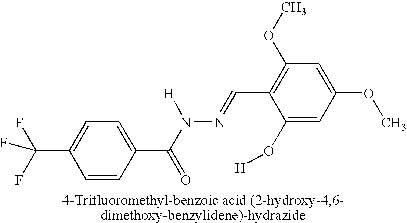 | CGP02-16 | 77% |
| 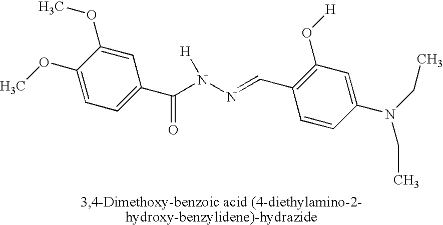 | CGP02-17 | 88% |
| 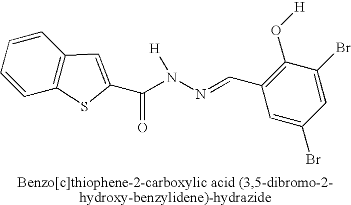 | CGP02-18 | 78% |

1) A compound with the following general formula (I): 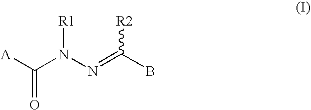
in which:
R1 and R2, identical or different, are chosen from among a hydrogen atom, a linear or branched lower alkyl radical of 1 to 6 carbon atoms, a fluoroalkyl radical of 1 to 9 carbon atoms and of 3 to 7 fluoride atoms,
A represents an aromatic group of one or several cycles possibly comprising one or several heteroatoms,
B represents a possibly substituted phenyl group or a possibly substituted pyridine group.

2) a compound of formula (I) according to claim 1, characterised by the fact that B represents a group with the following formula (II): 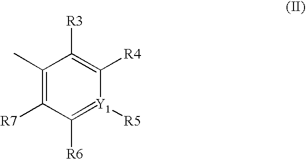
in which:
Y₁ is a carbon atom in order to form a phenyl nucleus or a nitrogen atom in order to form a pyridine nucleus,
R3, R4, R5, R6 and R7, either identical or different, are chosen from among: an atom of hydrogen, an atom of halogen and more particularly of fluoride, chloride and bromide, a group of formula —OH, —OR8 or —OCOR9, in which R8 and R9 represent a linear or branched lower alkyl radical of 1 to 6 carbons, an amino group —NH₂ or —N(r, r′) in which r and r′, either identical or different, represent a linear or branched lower alky radical, an aryl radical, or a heterocycle in which r and r′, taken together, form a heterocycle of variable size, preferably in the para position.

3) A compound of formula (I) according to claim 2, characterised by the fact that R3 is a group of formula —OR8 and at least two of the substituents R4, R5, R6 and R7 represent a hydrogen atom.

4) A compound of formula (I) according to claim 2, characterised by the fact that Y₁ is a carbon atom.

5) A compound of formula (I) according to claim 1, characterised by the fact that A represents a group with the following formula (III): 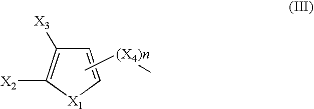
X₁ is chosen from among:
an oxygen atom and in this case the group of formula (III) is a 2-furanyl or 3-furanyl nucleus as a function of the position of the chain —(X₄)ₙ-acyl-hydrazide on the α or β carbons of this heterocycle,
a sulphur atom and in this case, the group of formula (III) is a 2-thiophene or 3-thiophen nucleus as a function of the position of the chain —(X₄)ₙ-acyl-hydrazide on the α or β carbons, this sulphur atom being capable of bearing an oxygen atom in order to form a sulphoxide or two oxygen atoms in order to form a sulphone.
a nitrogen atom and in this case, the group of formula (III) is a 2-pyrrol or 3-pyrrol nucleus as a function of the position of the acyl-hydrazide chain on the α or β carbons of this heterocycle, this nitrogen atom being capable of bearing a hydrogen atom, a lower alkyl radical of 1 to 6 carbon atoms, a fluoroalkyl radical with 1 to 6 carbon atoms and 3 to 7 fluoride atoms, an acyl radical —COR10 in which R10 represents a linear or branched alkyl chain of 1 to 6 carbons or an aryl or aralkyl radical,
X₂ and X₃, either identical or different, are chosen from among:
a hydrogen atom, a linear or branched lower alkyl chain of 1 to 6 carbon atoms, a fluoroalkyl radical with 1 to 6 carbon atoms and 3 to 7 fluoride atoms,
a halogen atom, preferentially a fluoride, chlorine or bromide atom,
a nitro —NO₂ group, an amino —NH₂ group or a —N(r, r′) group, in which r and r′, either identical or different represent a linear or branched lower alkyl radical, an aryl radical, or a heterocycle of variable size,
or furthermore X₂ and X₃ are included in an aromatic benzenic or aza-benzenic type cycle if this cycle comprises a nitrogen atom, in order to form an aromatic benzofuran heterocycle when X₁ is an oxygen atom, a benzopyrrol nucleus when X₁ is a nitrogen atom either free or substituted as above, a benzothiophene nucleus when X₁ is a sulphur atom either free or substituted as above or furthermore a pyridino type nucleus if an intracyclic nitrogen atom is present,
n is 0 or 1,
X₄, if present, represents a —CH₂—, —OCH₂—, or —CH═CH— group.

6) A compound according to claim 5, characterised by the fact that it is chosen from the group comprising:
N′-[(1E)-(2-hydroxy-4,6-dimethoxyphenyl)methylene]-1-benzothiophene-2-carbohydrazide,
(2Z)-3-(2-furyl)-N′-[(1E)-(2-hydroxy-4,6-dimethoxyphenyl)methylene] acrylohydrazide,
N′-[(1E)-(2-hydroxy-4,6-dimethoxyphenyl)methylene]-5-methylthiophene-2-carbohydrazide,
2-furancarboxylic acid (2-hydroxy-4,6-dimethoxy-benzylidene)-hydrazide (designated CGP02-07),
(1H-indol-3-yl) acetic acid (2-hydroxy-4,6-dimethoxybenzylidene)-hydrazide,
benzo[b]thiophene-2-carboxylic acid (3,5-dibromo-2-hydroxy-benzylidene)-hydrazide.

7) N′-[(1E)-(2-hydroxy-4,6-dimethoxyphenyl)methylene]-1-benzothiophene-2-carbohydrazide.

8) A compound of formula (I) according to claim 1, characterised by the fact that A represents a group with the following formula (V): 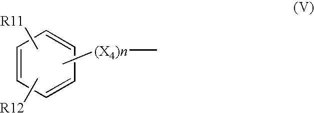
in which:
n is 0 or 1,
X₄, if present, represents a —CH₂—, —OCH₂—, or —CH═CH— group.
R11 and R12, either identical or different, in the ortho, meta or para positions in relation to the bond with —X₄— or in relation to the bond with —CO— when n is 0, are chosen from among a linear or branched-chain lower alkyl or aralkyl group of 1 to 6 carbon atoms or a fluoroalkyl radical with 1 to 6 carbon atoms and 3 to 7 fluoride atoms, a —OH, —OR13 or R13 radical represents a linear or branched-chain lower alkyl group of 1 to 6 carbon atoms, a halogen and more particularly of fluoride and specifically in this case, when R11 and R12 are fluoride atoms, they are in ortho on either side of the bond with —X₄— or the remainder —CO—,
where R12 represents a hydrogen atom and Rll represents a type —SO₂NH₂ sulphonamide group, in para in relation to the bond with —X₄— or the remainder —CO—,
or furthermore R11 represents a hydrogen atom and R12 represents a -Ophenyl group in ortho in relation to the bond with —X₄— or the remainder —CO—,

9) A compound of formula (I) according to claim 8, characterised by the fact that it is chosen from the group comprising:
(4-dimethylamino-N′-[(1E)-(2-hydroxy-4,6-dimethoxyphenyl)methylene]benzohydrazide,
2-phenethylbenzoic acid (2-hydroxy-4,6-dimethoxy-benzylidene)-hydrazide,
N-[3-2-hydroxy-4,6-dimenthoxy-benzylidene-hydrazinocarbonyl)-phenyl]-propionamide,
(3-chloro-phenoxy)-acetic acid (2-hydroxy-4,6-dimethoxybenzylidene)-hydrazide,
2-phenoxy-benzoic acid (2-hydroxy-4,6-dimethoxybenzylidene)-hydrazide,
2,6-difluorobenzoic acid (2-hydroxy-4,6-dimethoxybenzylidene)-hydrazide,
4-trifluoromethylbenzoic acid (2-hydroxy-4,6-dimethoxy-benzylidene)-hydrazide.
3,4-dimethoxybenzoic acid (4-diethylamino-2-hydroxy-benzylidene)-hydrazide

10) A compound of formula (I) according to claim 1, characterised by the fact that A represents a group with the following formula (VII): 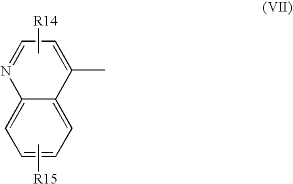
in which:
R15 is chosen from among an atom of hydrogen, an atom of halogen and more particularly of fluoride, chloride or bromide, a group of formula —OH, —OR16, in which R16 represents a linear or branched chain lower alkyl radical of 1 to 6 carbons or a fluoroalkyl radical with 1 to 6 carbon atoms and 3 to 7 fluoride atoms and more particularly a trifluoromethyl radical CF₃, R15 being positioned at one of the four remaining free sites of the 3-oxo-3,4-dihydro-benzothiazin-yl bicyclic aromatic part,
R14 represents a linear or branched alkyl radical of 1 to 6 carbons and more particularly a cyclopropyl radical.

11) A compound of formula (I) according to claim 10, characterised by the fact that R14 is in position 2 of the quinoline group and A represents a group of the following formula (VII′):

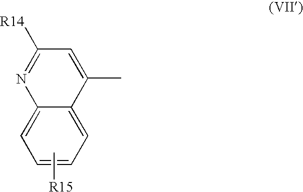

12) A compound of formula (I) according to claim 10, characterized by the fact that it is 2-cyclopropylquinoline-4-carboxylic acid (2-hydroxy-4,6-dimethoxy-benzylidene)-hydrazide.

13) A compound of formula (I) according to claim 1, characterised by the fact that A represents a group of the following formula (IX): 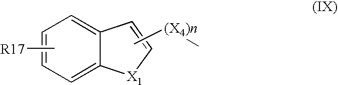
in which:
X₁ and X₄ have the same meaning as above,
n is 0 or 1,
R is chosen from among:
a hydrogen atom, a linear or branched lower alkyl radical of 1 to 6 carbon atoms, a fluoroalkyl radical of 1 to 6 carbon atoms and 3 to 7 fluoride atoms,
a halogen atom, preferentially an atom of fluoride, chlorine or bromide,
a group OR′ for which linear or branched lower R′ of 1 to 6 carbon atoms, a fluoroalkyl radical of 1 to 6 carbon atoms and 3 to 7 fluoride atoms.

14) A salt of a compound according to claim 1 with a pharmaceutically acceptable acid.

15) A pharmaceutical composition comprising as an active agent at least one compounds according to claim 1.

16) A composition according to claim 15, characterised by the fact that it is intended for treatment and/or prevention of diseases associated with lipid metabolism disorders.

17) A composition according to claim 15, characterised by the fact that it is intended for treatment and/or prevention of cardiovascular diseases.

18) A composition according to claim 15, characterised by the fact that it is intended for treatment and/or prevention of a disease chosen from the group including atherosclerosis, arterial restenosis, obesity, type II diabetes mellitus, cerebral ischaemia, epatic steatosis, hypercholesterolaemia, hypertriglyceridaemia, dyslipoproteinaemia, hylomicronaemia, lipodystrophy, hyperglycaemia and atherosclerosis.

19) (canceled)
